(19) United States
(12) Patent Application Publication
(10) Pub. No.: US 2014/0081160 A1
(43) Pub. Date: Mar. 20, 2014

(54) Flexible Multi-point Pulse Sensor

(71) Applicant: Jiannan Xiang, Suwanee, GA (US)

(72) Inventor: Jiannan Xiang, Suwanee, GA (US)

(21) Appl. No.: 13/623,179

(22) Filed: Sep. 20, 2012

(51) Int. Cl. A61B 5/024; A61B 5/0245

(52) U.S. Cl. CPC A61B 5/02444; A61B 5/0245
USPC 600/500; 29/622

(57) ABSTRACT

A flexible multi-point pulse sensing device is a kind of pulse detection device using capacitive sensor array. Characterized by simple structure, flexible material, high sensitivity, stability and precision, it makes possible to be applied to and coordinated with watch-type medical device, which can achieve convenient detection and monitoring of physiological parameters such as pulse rate and pressure at any time or place. It enables remote health monitoring a daily pulse changes without going to hospital every day.

### CROSS-REFERENCE TO RELATED APPLICATIONS

Not Applicable.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISC APPENDIX

Not Applicable.

### BACKGROUND OF THE INVENTION

Pulse is one of the important parameters of human body. It can reflect the pressure, relaxing, or other physiological status parameters. It's helpful to know health status of human body through measurement of pulse. Western medical pulse measuring device has been for several hundred years of history. With the progress of science and technology, especially the development of sensor technology, the use of sensor for the measurement of the body parameters such as pulse brought more convenience that ever before. It can realize miniaturization of measurement device, precision of measurement data, and visualization of testing data through combination of the test equipment and computer technology.

Sensor is a device that can convert physical quantities such as pressure, electric potential, and electric current into the electrical that can be identified, and then measure data. It mainly includes resistance sensor, piezoelectric sensor and capacitive sensor; all three kinds are available in the testing device of pulse or other physiological parameters. In the existing technology, the use of resistance sensor in measurement device is not high in accuracy. The device with use of piezoelectric sensor is high in accuracy. But it can't realize miniaturization of sensor and portability of device itself. Although piezoelectric sensor can be miniaturized by using semiconductor manufacturing process—the use of piezoelectric elements and pressure sensitive diode or pressure sensitive transistors formed by semiconductor strain gauge as pressure sensor, it costs high and the sensor made out is rigid and hard to achieve bending deformation. So it does not apply to the needs of flexible applications. However, using capacitive elements as sensing method has advantage of simple structure.

The specific comparison results of the three different kinds of sensors are shown below.

| | Capacitive | Resistance | Piezoelectric |
|---|---|---|---|
| Characteristics | sensor | sensor | sensor |
| Max measuring | Good | Good | Good |
| range | | | |
| Sensitivity | Excellent | Bad | Good |
| Min element size | Good | Excellent | Bad |
| Repeatability | Excellent | Bad | Good |
| Temperature | Excellent | Excellent | Bad |
| stability | | | |
| Design sensitivity | Excellent | Excellent | Good |

It's widely-know technology to determine arterial pressure variation through sensors. Place the sensor unit on the artery surface of human body and feel the tension changes of artery to determine the pressure state inside the blood vessels. Usually, the artery diameter is 1.2 mm-3.5 mm. In order to accurately check the pressure changes caused by artery, you need try to place the sensor on the right top of the artery. It's easy for the large sensor to achieve this goal, but it's hard to realize the miniaturization of its matched device and uncomfortable when used. What's worse, the artery pressure distribution changes along with extension directions of blood vessels; it's impossible to accurately determine the artery pressure distribution with sensor of large size. Therefore, the minute sized sensor is preferred to realize the goals mentioned above. Usually several miniature sensor units are arranged on the right top of extension directions of blood vessels orthogonally, as this arrangement will ensure that at least one sensor unit is one the top of artery in the measurement.

The documents of China's patent application number: 200510083261 released a kind of device that use capacitive sensor and the tension principle to determine pulse tension. In details, several miniature sensor units are arranged on the right top of extension directions of blood vessels orthogonally to determine the inside arterial pressure. This device contains the upper electrode, the lower electrode, and the base component which functions as the support between the electrodes. The electrode is made of copper foil, while the gasket is made by the silicone rubber. The upper electrode and the lower electrode are arranged crossly, and overlapped part forms the capacitive elements. When using it, wear the wrist with the detection device with capacitive sensor, and ensure your artery is right below the sensor unit. Press the device to make sure the sensor is close to your skin. Thus the inside arterial pressure variation is detected and calculated through the voltage change of capacitance elements.

The patent's problem is that, due to the material, size and shape limit of sensor elements and the support, the whole detection device is large in size and rigid in material. And you need to press it when use it. It's inconvenient and uncomfortable for users to use it, and it's not easy to carry. And the signal and area ratio (S/A) of the proposed polygonal-shaped sensor elements is small, and therefore the precision is vulnerable to the influence of the noise signals and can not meet the requirements of the later calculation and analysis.

In order to satisfy the convenient monitoring for the users at any time or place, especially the needs of inside arterial pressure, a miniature, and portable, precise and comfortable device is needed.

### BRIEF SUMMARY OF THE INVENTION

The present invention relates to a medical device and monitoring technology, especially involving the use of capacitive sensor array in pulse detection.

The invention provides a kind of capacitive sensor, including flexible substrate; the first electrode formed by sensing elements and solidified on the flexible substrate; the second electrode woven by metal material and fiber material; and the capacitive sensing unit—the cross section of the first and second electrode. The invention also provides a flexible multi-point pulse sensing device, including data wire; the capacitive sensor as mentioned above; and analog-to-digital conversion device installed on the flexible substrate.

The advantages of the invention lie in its simple structure and high sensitivity. Its flexible material makes it possible to attach to watch-type medical equipment strap. So it has characteristics of comfortable, convenient, flexible and low manufacturing cost.

### BRIEF DESCRIPTION OF THE SEVERAL VIEWS OF THE DRAWING

Having thus described the invention in general terms, reference will now be made to the accompanying drawings, which are not necessarily drawn to scale, and wherein:

FIG. 1: Schematic drawing of the capacitive sensor

FIG. 2: Schematic drawing of the sensor device

FIG. 3: Schematic plan and side drawing of sensor device's second electrode

FIG. 4: Schematic drawing of the first electrode in Embodiment 1

FIG. 5: Schematic drawing of the first electrode in Embodiment 2

FIG. 6: Schematic drawing of the first electrode in Embodiment 3

FIG. 7: Schematic drawing of the first electrode in Embodiment 4

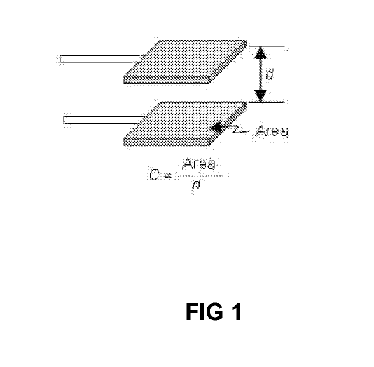

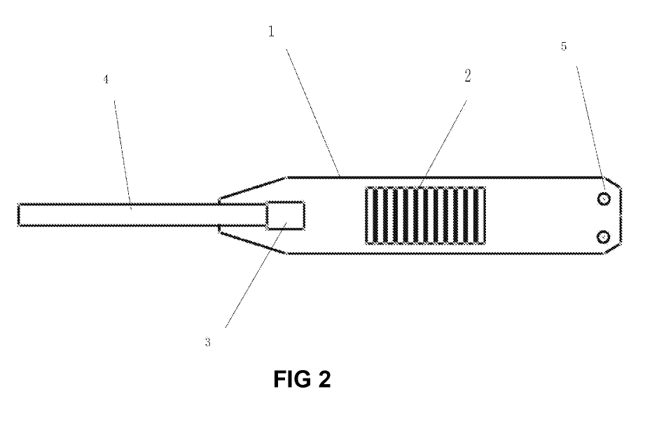

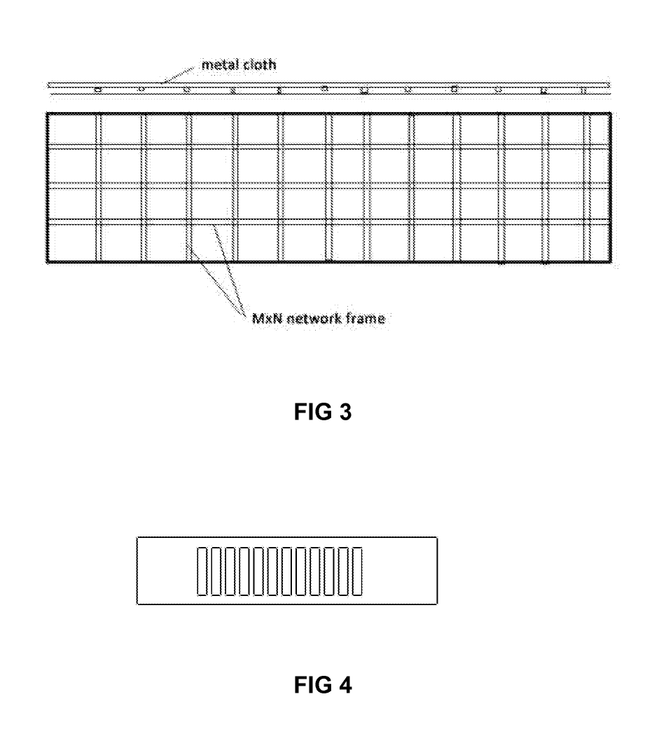

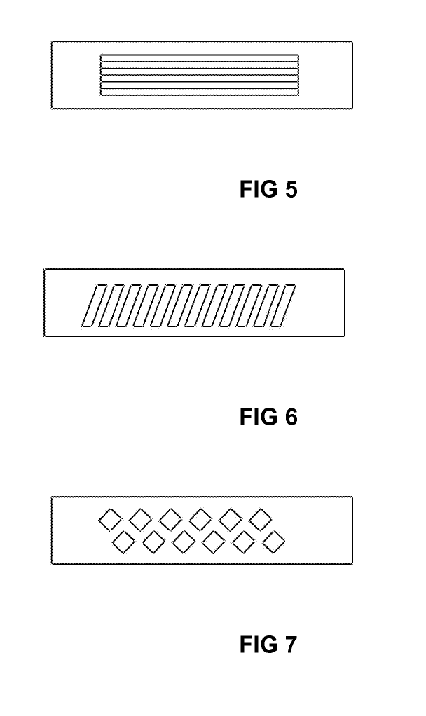

### DETAILED DESCRIPTION OF THE INVENTION

In order to solve the problems of existing technologies, the present invention provides a multi-point pulse sensing device with flexible capacitive sensor array. It has the characteristics of simple structure, low manufacturing cost, precise measurement results, high stability, and more suitable for flexible structure such as wristbands. At the same time, it can accurately detect the pulse pressure. Coordinated with watch-style medical device, the invention makes it possible and convenient for users to monitor their physiological parameters such as pulse pressure at any time or place.

The present invention provides a making procedure of the flexible capacitive sensor, including:
a) Solidify the regularly arranged sensing elements onto the flexible substrate (FPCB) to form the first electrode of the capacitive sensor;
b) Mix and weave the metallic wire/foil and fiber material to form the second electrode facing the first one.

The procedure to connect and fix the two electrodes is included, and the above-mentioned metallic wire/foil to form the second electrode needs to be in reticular cross arrangement.

The invention provides a capacitive sensor, including:
Flexible substrate (FPCB);
The first electrode formed by sensing elements and solidified on the flexible substrate;
The second electrode woven by metal material and fiber material;

And the cross section of the mentioned first and second electrode forms the capacitive sensing unit.

The above-mentioned metallic wire/foil to form the second electrode needs to be in reticular cross arrangement.

The mentioned flexible substrate needs to have at least one connection and fixing device.

The invention also provides a flexible multi-point pulse sensing device, characterized by: including:
Data wire;
The capacitive sensor as mentioned above;
Analog-to-digital conversion device installed on the flexible substrate.

The area of the above-mentioned first electrode is 20 mm×15 mm.

The width of the above-mentioned flexible substrate is 18 mm.

The advantages of the invention lie in its simple structure and high sensitivity. Its flexible material makes it possible to attach to watch-type medical equipment strap. So it has characteristics of comfortable, convenient, flexible and low manufacturing cost.

### DESCRIPTION OF THE PREFERRED EMBODIMENTS

The capacitive sensor used in this invention to detect arterial pressure is based on the basic electrical properties of capacitance. As showed in the appended drawing 1, the two electrodes of the capacitance are separated by airspace d. If the airspace reduces, the capacitance C rises; conversely, capacitance C declines. Based on this principle, we apply capacitance sensor to pulse detection. A method to make new capacitance sensor is used, that is, one electrode of the capacitance is mixed woven by electrode material and fiber material, and cloth can be used as a touch pad contacting human body. The other electrode is capacitance elements solidified on the flexible substrate which are calibrated into array configuration. The two electrodes match and form the capacitance sensor, while the cross section of the two electrodes forms the capacitive sensing unit which will be used to test the capacitance change.

When this sensor device is fixed on the arm, the fiber material with electrode will contact the arm surface, and the sensor array on the flexible substrate will be roughly on the top of artery. When arterial pressure presses the cloth, it will change the distance of capacitance as well as the electrical potential changes between the capacitive sensing units. The detected electrical potential changes will be converted into digital signals through analog-to-digital conversion device and then sent to watch-type medical device. After further analysis and process of these digital signals, the human body's parameters such as arterial pressure will be detected. At present, the reliability and sensitivity of this flexible sensor can be realized by the existing manufacturing technology. In addition, the flexibility of the invention helps to realize comfortable, stretchable, industrialized and other mixed design.

### Embodiment 1

This embodiment is an implementation plan to apply the invention to watch-type medical equipment. The sensor device, shown in the appended drawing 2, including: flexible substrate (FPCB) (1), the first electrode solidified on the flexible substrate (2), analog-to-digital conversion device (3), as well as the cloth containing the second electrode shown in drawing 3. Flexible substrate (1) bears the weight of the first electrode (2 ) and analog-to-digital conversion device (3), and attaches to the wristband of the watch-type medical equipment. The first electrode (2) is solidified onto the flexible substrate (1). It contains M regularly arranged capacitance elements. Here M is greater than the natural number 2. The first electrode is arranged roughly parallel with artery direction. Every capacitance element's width is 1 mm-2 mm, and the distance between each is 0.1 mm-0.3 mm.

Such arrangement can ensure there is at least one capacitance element on the right top of the artery, so as to make sure the accuracy of measurement. One end of the flexible substrate is installed with the analog-to-digital conversion device (3), whose type is AD7147. It's used to collect the analog signals sent by capacitance sensor, and convert the signals into digital ones. The digital signals will be transmitted to the watch-type medical equipment through data wire (4). Flexible substrate (1) can be equipped with at least one connection and fixation device used to connect and fix the flexible substrate and the cloth in the first and second electrode.

The appended drawing 3 shows an arrangement method of the second electrode. Wove the metallic wire or foil into a mesh, and then mix with the fiber material to form the second electrode. The purpose to use fiber is its function of making interval between the first and the second electrode. The cross section of the first and second electrode forms the capacitive sensing unit.

In the case, the minimum working voltage of the sensor device is 3.3V, and the maximum current is 1 mA. The array is made of 12 capacitance elements, forming the area of 20 mm×15 mm. The width of the flexible substrate is 18 mm, roughly matched with the width of the watchband. The whole sensor device is installed onto the watchband of the medical equipment. The sensor receives the power and commands from the medical equipment through data wire. When receives the commands of start, it starts to work, collect and sent data. When receives the commands to stop, the power supply to the sensor is off and it stops working.

### Embodiment 2

In this embodiment, the model and material of the parts and the implementation plan are the same as the embodiment 1. The only difference is that, as shown in the appended drawing 5,

The first electrode is still arranged roughly parallel with artery direction, and every capacitance element's width is 1 mm-2 mm, however, the distance between each elements enlarges and the length extends more. This kind of arrangement can ensure more than one capacitance element on the right top of the artery.

### Embodiment 3

In this embodiment, the model and material of the parts and the implementation plan are the same as the embodiment 1. The only difference is that, as shown in the appended drawing 6, the capacitance elements to form the first electrode are in parallel arrangement but at an angel of 45 degrees to the bottom of flexible substrate. This kind of arrangement can ensure there is at least one capacitance element on the right top of the artery to work. The advantage is that the airspace between two capacitance elements can be larger so as to make it convenient for designing, manufacturing and using.

### Embodiment 4

In this embodiment, the model and material of the parts and the implementation plan are the same as the embodiment 1. The only difference is that, as shown in the appended drawing 7, the capacitance elements to form the first electrode are shaped in 2 mm×2 mm square and arranged in matrix form; the distance between each two elements is still 0.1-0.3 mm. The advantage is that it improves the ratio of S/A(Signal to Area) and provides better measurement results.

The above four embodiments are only the preferred implementation methods of this invention. It should points out that the common technicians of this technology field can also make some improvements and decorates on the premise that they comply with the principle of invention. These improvements and decorates shall be regarded as the protection scope of the invention, for example, the changes in sensor size.

What I claim as my invention is:

1. A making procedure of the flexible capacitive sensor is characterized by:
a) Solidify the regularly arranged sensing elements onto the flexible substrate (FPCB) to form the first electrode of the capacitive sensor;
b) Mix and weave the metallic wire/foil and fiber material to form the second electrode facing the first one.

2. According to the claim 1, the making procedure of this flexible capacitive sensor is characterized by: the procedure to connect and fix the two electrodes is included.

3. According to the claim 1, the making procedure of this flexible capacitive sensor is characterized by: the metallic wire/foil to form the second electrode needs to be in reticular cross arrangement.

4. A capacitive sensor is characterized by: including:
Flexible substrate;
The first electrode formed by sensing elements and solidified on the flexible substrate;
The second electrode woven by metal material and fiber material;
And the capacitive sensing unit—the cross section of the first and second electrode.

5. According to the claim 4, the capacitive sensor is characterized by: the first electrode contains m regularly arranged capacitance elements. Here m is greater than the natural number 2.

6. According to the claim 4, the capacitive sensor is characterized by: the metallic wire/foil and fiber material to form the second electrode needs to be woven reticular.

7. According to the claim 4, the capacitive sensor is characterized by: the mentioned flexible substrate needs to have at least one connection and fixing device.

8. A flexible multi-point pulse sensing device is characterized by: including Data wire;
The capacitive sensor as mentioned in claim 4,5,6,7 above;
And analog-to-digital conversion device installed on the flexible substrate.

9. According to the claim 8, the flexible multi-point pulse sensing device is characterized by: the first electrode area is 20 mm×15 mm.

10. According to the claim 8, the flexible multi-point pulse sensing device is characterized by: the width of the flexible substrate is 18 mm.
